(19) United States
(12) Patent Application Publication
(10) Pub. No.: US 2006/0280711 A1
(43) Pub. Date: Dec. 14, 2006

(54) Process for treating marionette lines

(75) Inventors: Marc Cornell, Jackson, NJ (US); Isabelle Hansenne, Westfield, NJ (US); Hani Fares, Somerset, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/152,707

(22) Filed: Jun. 14, 2005

(51) Int. Cl. A61K 31/375; A61K 8/89

(52) U.S. Cl. 424/70.12; 424/401; 514/474

(57) ABSTRACT

A process for treating marionette lines damaged by age, sun exposure and pollution involving contacting the marionette lines with a composition containing: (a) from about 1 to about 20% by weight of ascorbic acid; (b) from about 30 to about 80% by weight of a nonaqueous polar organic solvent; and (c) from about 20 to about 60% by weight of a nonaqueous nonpolar organic solvent, all weights being based on the weight of the composition.

### BACKGROUND OF THE INVENTION

It is well known that aging of the skin is due, at least in part, to continual stretching and contraction of both the dermal and epidermal layers of the skin and disruption of the collagen bundles which provide support to the epidermis. Collagen consists of long elastic polypeptide fibers interconnected by bridges which provide the cohesion and stability of connective tissue. This enables collagen to act as an elastic tissue in every direction and retain water. Collagen aging manifests itself as a break in connection between the collagen fibers. Age, severe weather, and pollution accelerate the breaks and slow down renewal of the collagen structure.

Ascorbic acid, or Vitamin C, has many known biological functions. The L-ascorbic acid isomer is biologically active and is known to stimulate the synthesis of collagen, act as a free radical scavenger, and minimize lipid peroxidation and other forms of cellular damage associated with aging. Ascorbic acid is a white, odorless, crystalline solid having the empirical formula C6H8O6, a molecular weight of about 176, and corresponds to the formula:

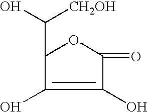

Another active ingredient typically used to reverse the damage caused by age, severe weather and pollution is retinol. Retinoic acid, which is derived from retinol, is known to activate skin cell metabolism resulting in collagen production.

Conventional anti-aging formulations based on ascorbic acid and retinol as the active ingredient have been found to have an effect on wrinkles such as, for example, orbital wrinkles and crows feet. Such formulations, however, have had minimal, if any, effect on marionette lines which are defined as lines which go down on either side of a person's mouth, also known as “oral commissures.”

### SUMMARY OF THE INVENTION

The present invention is thus directed to a process for treating marionette lines which have been damaged by age, severe weather and pollution comprising contacting the marionette lines with a composition containing:

from 1 to 20% by weight of ascorbic acid;

from 40 to 80% by weight of a nonaqueous polar organic solvent; and

from 20 to 60% by weight of a nonaqueous nonpolar organic solvent, all weights being based on the weight of the composition.

### DETAILED DESCRIPTION

Other than in the operating examples, or where otherwise indicated, all numbers expressing quantities of ingredients and/or reaction conditions are to be understood as being modified in all instances by the term “about.”

### DEFINITIONS

The term “polar solvent” means one which is capable of dissolving at least 2 weight percent or more of ascorbic acid at room temperature (generally 25° C.)

The term “nonpolar solvent” means one which is capable of dissolving less than 2 weight percent of ascorbic acid at room temperature.

The term “anhydrous” means that no substantial amount of water is present in the compositions of the invention.

The compositions of the invention may be liquid, semi-solid, or solid at room temperature. The compositions exist in an anhydrous emulsion form. The term “emulsion” is generally used in the cosmetic art to mean water-in-oil or oil-in-water emulsions. However, the compositions of the invention are anhydrous emulsions wherein one anhydrous phase (‘the dispersed phase’) is dispersed into another anhydrous phase (“the continuous phase”). In the anhydrous emulsions of the invention, the ascorbic acid dissolved into the nonaqueous polar organic solvent forms the dispersed phase. The nonaqueous nonpolar organic solvent forms the continuous phase.

All percentages mentioned herein are percentages by weight unless otherwise indicated.

Ascorbic Acid

The term “ascorbic acid” when used in accordance with this invention means L-ascorbic acid, D-ascorbic acid, and derivatives thereof. Ascorbic acid may be employed in an amount of from 1 to 20% by weight, preferably from 5 to 15% by weight, and more preferably from 5 to 10% by weight, based on the weight of the composition.

The Nonaqueous Polar Organic Solvent

The anhydrous emulsions of the invention contain from 30 to 80% by weight, preferably from 40 to 60% by weight, and more preferably from 40 to 50% by weight, based on the weight of the total composition, of a nonaqueous polar organic solvent. A variety of nonaqueous polar organic solvents are suitable for use in the dispersed phase of the anhydrous emulsion. Examples are as follows.

Polyols

Polyols are suitable nonaqueous polar organic solvents. For purposes of this specification, polyols are defined as compounds which contain three or more hydroxyl groups per molecule. Examples of suitable polyols include fructose, glucamine, glucose, glucose glutamate, glucuronic acid, glycerin, 1,2,6hexanetriol, hydroxystearyl methylglucanine, inositol, lactose, malitol, mannitol, methyl gluceth-10, methyl gluceth-20, methyl glucose dioleate, methyl glucose sesquicaprylate/sesquicaprate, methyl glucose sesquicocoate, methyl glucose sesquiisostearate, methyl glucose sesquilaurate, methyl glucose sesquistearate, phytantriol, riboflavin, sorbeth-6, sorbeth-20, sorbeth-30, sorbeth-40, sorbitol, sucrose, thioglycerin, xylitol, and mix thereof. An especially preferred polyol is glycerin.

Polymeric or Monomeric Ethers

Also suitable as the nonaqueous polar organic solvent are homopolymeric or block copolymeric liquid ethers. Polymeric ethers are preferably formed by polymerization of monomeric alkylene oxides, generally ethylene or propylene oxides. Examples of such polymeric ethers include PEG, PPG, and derivatives thereof.

Other examples of suitable polymeric ethers include polyoxypropylene polyoxyethylene block copolymers. Such compounds are sold under the CTFA name Meroxapol 105, 108, 171, 172, 174, 178, 251, 252, 254, 255, 258, 311, 312, and 314.

Mono- and Dihydric Alcohols

Also suitable for use as to the nonaqueous polar organic solvent are mono- and dihydric alcohols of the general formula R(OH)n where n is 1 or 2 and R is a substituted or unsubstituted saturated C2-10, preferably C1-8 alkyl, or a substituted or unsubstituted alicyclic, bicyclic, or aromatic ring, with the substituents selected from halogen, alkoxy, hydroxy, and so on. Examples of suitable alcohols include monohydric alcohols such as ethanol, isopropanol, hexyldecanol, benzyl alcohol, propyl alcohol, and isopropyl alcohol, as well as dihydric alcohols such as hexylene glycol, diethylene glycol, ethylene glycol, propylene glycol, 1,2-butylene glycol, triethylene glycol, dipropylene glycol, and mixtures thereof.

Sorbitan Derivatives

Sorbitan derivatives, which are defined as ethers or esters of sorbitan, are also suitable polar solvents. Examples of suitable sorbitan derivatives are the Polysorbates, which are defined as stearate esters of sorbitol and sorbitan anhydrides, such as Polysorbate 20, 21, 40, 60, 61, 65, 80, 81, and 85. Also suitable are fatty esters of hexitol anhydrides derived from sorbitol, such as sorbitan trioleate, sorbitan tristearate, sorbitan sesquistearate, sorbitan stearate, sorbitan palmitate, sorbitan oleate, and mixtures thereof.

The Nonaqueous Nonpolar Organic Solvent

The anhydrous emulsions of the invention contain from 20 to 60% by weight, preferably from 20 to 50% by weight, and more preferably from 30 to 40% by weight, based on the weight of the total composition, of a nonaqueous nonpolar organic solvent as the continuous phase. A variety of nonaqueous nonpolar organic solvents can be used in the compositions of the invention.

Silicones

Silicones are suitable nonpolar compounds. The silicones may be volatile or non-volatile. The term “volatile” means that the silicone has a measureable vapor pressure, i.e. a vapor pressure of at least 2 mm. of mercury at 20° C. If volatile, the silicone generally will have a viscosity of 0.5 to 25 centistokes at 25° C. Suitable volatile silicones include cyclic silicones, linear silicones, or mixtures thereof.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, and mixtures thereof.

The silicone may also be nonvolatile, and in particular water insoluble nonvolatile silicones. The term “nonvolatile” means that the silicone has a vapor pressure of less than 2 mm. of mercury at 20° C. A variety of silicones fit this definition including dimethicone, phenyl trimethicone, diphenyl dimethicone, methicone, hexadecyl methicone, stearoxydimethicone, stearyl dimethicone, cetyl dimethicone, and so on.

Cyclomethicone is the preferred silicone for use in the compositions of the invention.

Esters

In addition to the sorbitan esters, other esters are also suitable as the nonaqueous nonpolar organic solvent. In general such esters have the formula RCO—OR wherein each R is independently a C1-25 straight or branched chain saturated or unsaturated alkyl, alkylcarbonyloxyalkyl, or alkoxycarbonylalkyl, aryl, which may be substituted or unsubstituted with halogen, hydroxyl, alkyl, and the like.

Examples of suitable esters include alkyl acetates, alkyl behenates, alkyl lactates, alkyl benzoates, alkyl octanoates, alkyl salicylates, and in particular C12-15 alkyl benzoate. Examples of further esters are set forth on pages 502-506 of the CTFA Cosmetic Ingredient Handbook, Second Edition, 1992, which is hereby incorporated by reference.

Fats and Oils

Fats and oils are also suitable as the nonaqueous nonpolar organic solvent. Preferably these materials are liquids or semi-solids at room temperature. They are generally defined as glyceryl esters of fatty acids (triglycerides), as well as the synthetically prepared esters of glycerin and fatty acids. Examples of such materials include oils such as apricot kernel oil, avocado oil, canola oil, olive oil, sesame oil, peanut oil, soybean oil, trilinolenin, trilinolein, trioctanoin, tristearin, triolein, sesame oil, rapeseed oil, sunflower seed oil, and so on.

Fatty Acids

Fatty acids are also suitable as the nonaqueous nonpolar organic solvent in the compositions of the invention. Preferably the fatty acids are liquid or semi solid at room temperature. Fatty acids are the carboxylic acids obtained by hydrolysis of animal or vegetable fats and oils. Carboxylic acids having alkyl chains shorter than about seven carbon atoms are not generally considered fatty acids. Fatty acids have the general structure R—COOH where R is a straight or branched chain saturated or unsaturated C7-65 alkyl. Examples of suitable fatty acids include arachidic acid, arachidonic acid, behenic acid, capric acid, caproic acid, caprylic acid, coconut acid, corn acid, cottonseed acid, hydrogenated coconut acid, hydroxystearic acid, lauric acid, linoleic acid, linolenic acid, linseed acid, myristic acid, oleic acid, palmitic acid, palm kernel acid, soy acid, tallow acid, and the like.

Fatty Alcohols

Fatty alcohols may also be used as the nonaqueous nonpolar organic solvent. Fatty alcohols are generally made by reducing the fatty acid —COOH group to the hydroxyl function. They generally have the formula RCH20H. Examples of fatty alcohols are behenyl alcohol, C9-11 alcohol, C12-13 alcohol, C12-15 alcohol, C12-16 alcohol, caprylic alcohol, cetearyl alcohol, cetyl alcohol, coconut alcohol, decyl alcohol, lauryl alcohol, myristyl alcohol, oleyl alcohol, and the like.

Hydrocarbons

Hydrocarbons are also good nonaqueous nonpolar organic solvents in accordance with the invention. Examples of suitable hydrocarbons include C7-60 isoparaffins, ethane, heptane, hexane, hydrogenated polyisobutene, isobutane, isododecane, isoeicosane, isohexadecane, isopentane, microcrystalline wax, mineral oil, mineral spirits, paraffin, petrolatum, petroleum distillates, squalene, polyethylene, and mixtures thereof. Preferred hydrocarbons are mineral oil and polyethylene.

Lanolin and Lanolin Derivatives

Also suitable as the nonaqueous nonpolar organic solvent are lanolin and derivatives thereof. Examples of such materials include acetylated hydrogenated lanolin, acetylated lanolin alcohol, laneth, lanolin acid, lanolin oil, lanolin alcohol, lanolin wax, and so on.

Other Ingredients

It may also be desired to include certain other ingredients in the anhydrous emulsions of the invention, such as surfactants, waxes, colorants, preservatives, and so on.

Surfactants

Silicone Surfactants

The compositions may contain 0.1-15%, preferably 0.5-10%, more preferably 1-8% by weight of the total composition of one or more surfactants. The term “surfactant” is defined, in accordance with the invention, as a compound having at least one hydrophilic moiety and at least one lipophilic moiety. The surfactants may be silicone surfactants (also referred to as organosiloxane emulsifiers) or organic surfactants.

Suitable silicone surfactants used in the compositions of the invention may be liquid or solid at room temperature and are generally a water-in-oil or oil-in-water type surfactants which are preferably nonionic, having an Hydrophile/Lipophile Balance (HLB) of 2 to 18. Preferably the organosiloxane is a nonionic surfactant having an HLB of 2 to 12, preferably 2 to 10, most preferably 4 to 6. The HLB of a nonionic surfactant is the balance between the hydrophilic and lipophilic portions of the surfactant and is calculated according to the following formula:
HLB=20(1−S/A)
where S is the saponification number of the surfactant and A is the acid number of the surfactant.

The silicone surfactant or emulsifier used in the compositions of the invention is a polymer containing a polymeric backbone including repeating siloxy units that may have cyclic, linear or branched repeating units, e.g. di(lower)alkylsiloxy units, preferably dimethylsiloxy units. The hydrophilic portion of the organosiloxane is generally achieved by substitution onto the polymeric backbone of a radical that confers hydrophilic properties to a portion of the molecule. The hydrophilic radical may be substituted on a terminus of the polymeric organosiloxane, or on any one or more repeating units of the polymer. In general, the repeating dimethylsiloxy units of modified polydimethylsiloxane emulsifiers are lipophilic in nature due to the methyl groups, and confer lipophilicity to the molecule. In addition, longer chain alkyl radicals, hydroxy-polypropyleneoxy radicals, or other types of lipophilic radicals may be substituted onto the siloxy backbone to confer further lipophilicity and organocompatibility. If the lipophilic portion of the molecule is due in whole or part to a specific radical, this lipophilic radical may be substituted on a terminus of the organosilicone polymer, or on any one or more repeating units of the polymer. It should also be understood that the organosiloxane polymer in accordance with the invention should have at least one hydrophilic portion and one lipophilic portion.

The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, sulfonates, sulfates, phosphates, or amines.

The term “lipophilic radical” means an organic radical that, when substituted onto the organosiloxane polymer backbone, confers lipophilic properties to the substituted portion of the polymer. Examples of organic radicals which will confer lipophilicity are C1-40 straight or branched chain alkyl, fluoro, aryl, aryloxy, C1-40 hydrocarbyl acyl, hydroxy-polypropyleneoxy, or mixtures thereof. The C1-40 alkyl may be non-interrupted, or interrupted by one or more oxygen atoms, a benzene ring, amides, esters, or other functional groups.

The polymeric organosiloxane emulsifier used in the invention may have any of the following general formulas:
MxQy, or
MₓT_{y}, or
MDₓD′_{y}D″_{z}M

wherein each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof. T is a trifunctional siloxy unit having the empirical formula RSiO₁₅ or RSiO₁₅. Q is a quadrifunctional siloxy unit having the empirical formula SiO₂, and D, D′, D″, x, y, and z are as set forth below, with the proviso that the compound contains at least one hydrophilic radical and at least one lipophilic radical. Examples of emulsifiers used in the compositions of the invention are of the general formula:
MDₓD′_{y}D″_{z}M
wherein the trimethylsiloxy endcap unit is unsubstituted or mono-substituted, wherein one methyl group is substituted with a lipophilic radical or a hydrophilic radical. Examples of such substituted trimethylsiloxy endcap units include (CH₃)₂HPSiO, (CH₃)₂LPSiO, (CH₃)₂CH₂HPSiO, (CH₃)₂CH₂LPSiO, wherein HP is a hydrophilic radical and LP is a lipophilic radical. D, D′, and D″ are difunctional siloxy units substituted with methyl, hydrogen, a lipophilic radical, a hydrophilic radical or mixtures thereof. In this general formula:
x=0-5000, preferably 1-1000
y=0-5000, preferably 1-1000, and
z=0-5000, preferably 0-1000,
with the proviso that the compound contains at least one lipophilic radical and at least one hydrophilic radical. Examples of these polymers are disclosed in U.S. Pat. No. 4,698,178, which is hereby incorporated by reference.

Particularly preferred is a linear silicone of the formula:
MDₓD′_{y}D″_{z}M
wherein M=RRRSiO_{1/2}
D and D′=RR′SiO_{2/2}
D″=RRSiO_{2/2}
x, y, and z are each independently 0-1000,

where R is methyl or hydrogen, and R′ is a hydrophilic radical or a lipophilic radical, with the proviso that the compound contains at least one hydrophilic radical and at least one lipophilic radical.

Most preferred is wherein
M=trimethylsiloxy
D=Si[(CH₃)][(CH₂)ₙCH₃]O_{2/2} where n=1-40,
D′=Si[(CH₃)][(CH₂)ₒ—O—PE)]O_{2/2} where PE is (—C₂H₄O)ₐ(—C₃H₆O)_{b}H,
o=0-40, a=1-100 and b=1-100, and
D″=Si (CH₃)₂O_{2/2}.

Organosiloxane polymers useful in the compositions of the invention are commercially available from Goldschmidt Corporation under the ABIL tradename. The preferred polymer is cetyl dimethicone copolyol and has the tradename ABIL WE 09 or ABIL WS 08. The cetyl dimethicone copolyol may be used alone or in conjunction with other non-silicone organic emulsifiers. Preferred is where the cetyl dimethicone copolyol is in an admixture with other non-silicone organic emulsifiers and emollients. In particular, blends of 25-50% of the organosiloxane emulsifier, 25-50% of a non-silicone organic emulsifier, and 25-50% by weight emollients or oils are preferred. For example, the mixtures identified by the C.T.F.A. names cetyl dimethicone copolyol (and) polyglyceryl 4-isostearate (and) hexyl laurate, or cetyl dimethicone copolyol (and) polyglyceryl-3 oleate (and) hexyl laurate both work well. These blends contain approximately 25-50% of each ingredient, for example ABIL WE 09 contains approximately, by weight of the total ABIL composition, 25-50% cetyl dimethicone copolyol, 25-50%, polyglyceryl 4-isostearate, and 25-50% of hexyl laurate which is an emollient or oil.

Another type of preferred organosiloxane emulsifier suitable for use in the compositions of the invention are emulsifiers sold by Union Carbide under the Silwet™ trademark. These emulsifiers are represented by the following generic formulas:
(Me₃Si)_{y}−2[(OSiMe₂)ₓ/yO—PE]_{y}
wherein PE=—(EO)ₘ(PO)ₙR
R=lower alkyl or hydrogen
Me=methyl
EO is polyethyleneoxy
PO is polypropyleneoxy
m and n are each independently 1-5000
x and y are each independently 0-5000, and 8
wherein PE=—CH₂CH₂CH₂O(EO)ₘ(PO)ₙZ
Z=lower alkyl or hydrogen, and
Me, m, n, x, y, EO and PO are as described above,
with the proviso that the molecule contains a lipophilic portion and a hydrophilic portion. Again, the lipophilic portion can be supplied by a sufficient number of methyl groups on the polymer backbone.

A preferred organosiloxane emulsifier for use in the compositions of the invention is dimethicone copolyol.

Examples of other polymeric organosiloxane surfactants or emulsifiers include amino/polyoxyalkyleneated polydiorganosiloxanes disclosed in U.S. Pat. No. 5,147,578. Also suitable are organosiloxanes sold by Goldschmidt under the ABIL trademark including ABIL B-9806, as well as those sold by Rhone-Poulenc under the Alkasil tradename. Also, organosiloxane emulsifiers sold by Amerchol under the Amersil tradename, including Amersil ME-358, Amersil DMC-287 and Amersil DMC-357 are suitable. Dow Corning surfactants such as Dow Corning 3225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention.

Also suitable as surfactants are various organic surfactants such as anionic, nonionic, amphoteric, zwitterionic, or cationic surfactants.

The compositions of the invention comprise 0.5-20%, preferably 0.5-15%, more preferably 0.5-10%, of a surfactant. Suitable surfactants may be anionic, nonionic, amphoteric, or zwitterionic.

Anionic Surfactants

Anionic surfactants include alkyl and alkyl ether sulfates generally having the formula ROSO₃M and RO(C₂H₄O)ₓSO₋₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the compositions of the invention are water soluble salts of organic, sulfric acid reaction products of the general formula:
R₁—SO₃-M
wherein R₁ is chosen from the group consisting of a straight or branched chain, saturated aliphatic hydrocarbon radical having from about 8 to about 24 carbon atoms, preferably 12 to about 18 carbon atoms; and M is a cation. Examples of such anionic surfactants are salts of organic sulfuric acid reaction products of hydrocarbons such as n-paraffins having 8 to 24 carbon atoms, and a sulfonating agent, such as sulfur trioxide.

Also suitable as anionic surfactants are reaction products of fatty acids esterified with isethionic acid and neutralized with sodium hydroxide. The fatty acids may be derived from coconut oil, for example.

In addition, succinates and succinimates are suitable anionic surfactants. This class includes compounds such as disodium N-octadecylsulfosuccinate; tetrasodium N-(1,2-dicarboxyethyl)-N-octadecyl-sulfosuccinate; and esters of sodium sulfosuccinic acid e.g. the dihexyl ester of sodium sulfosuccinic acid, the dioctyl ester of sodium sulfosuccinic acid, and the like.

Other suitable anionic surfactants include olefin sulfonates having about 12 to 24 carbon atoms. The term “olefin sulfonate” means a compound that can be produced by sulfonation of an alpha olefin by means of uncomplexed sulfur trioxide, followed by neutralization of the acid reaction mixture in conditions such that any sulfones which have been formed in the reaction are hydrolyzed to give the corresponding hydroxy-alkanesulfonates. The alpha-olefin from which the olefin sulfonate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof such as the salts of Cl₀₋₂₀ fatty acids, for example coconut and allow based soaps. Preferred salts are ammonium, potassium, and sodium salts.

Still another class of anionic surfactants include N-acyl amino acid surfactants and salts thereof (alkali, alkaline earth, and ammonium salts). Examples of such surfactants are the N-acyl sarcosinates, including lauroyl sarcosinate, myristoyl sarcosinate, cocoyl sarcosinate, and oleoyl sarcosinate, preferably in sodium or potassium forms.

Nonionic Surfactants

The composition can contain one or more nonionic surfactants. Nonionic surfactants are generally compounds produced by the condensation of alkylene oxide groups with a hydrophobic compound. Classes of nonionic surfactants are:

(a) Long chain dialkyl sulfoxides containing one short chain alkyl or hydroxy alkyl radical of from about 1 to 3 carbon atoms and one long hydrophobic chain which may be an alkyl, alkenyl, hydroxyalkyl, or ketoalkyl radical containing from about 8 to 20 carbon atoms, from 0 to 10 ethylene oxide moieties, and 0 or 1 glyceryl moiety.

(b) Polysorbates, such as sucrose esters of fatty acids. Examples of such materials include sucrose cocoate, sucrose behenate, and so on.

(c) Polyethylene oxide condensates of alkyl phenols, for example the condensation products of alkyl phenols having an alkyl group of 6 to 20 carbon atoms With ethylene oxide being present in amounts of about 10 to 60 moles of ethylene oxide per mole of alkyl phenol.

(d) Condensation products of ethylene oxide with the reaction product of propylene oxide and ethylene diamine.

(e) Condensation products of aliphatic alcohols having 8 to 18 carbon atoms with ethylene oxide, for example a coconut alcohol/ethylene oxide condensate having 10 to 30 moles of ethylene oxide per mole of coconut alcohol, the coconut alcohol fraction having 10 to 14 carbon atoms.

(f) Long chain tertiary amine oxides such as those corresponding to the general formula:
R₁R₂R₃NO
wherein R₁ contains an alkyl, alkenyl or monohydroxyalkyl radical ranging from about 8 to 18 carbon atoms in length, from 0 to about 10 ethylene oxide moieties, and from 0 to about 1 glyceryl moiety and R₂ and R₃ are each alkyl or monohydroxyalkyl groups containing from about 1 to about 3 carbon atoms.

(g) Long chain tertiary phosphine oxides corresponding to the general formula:
RR₁R₂PO
wherein R contains an alkyl, alkenyl, or monohydroxyalkyl radical having 8 to 18 carbon atoms, from 0-10 ethylene oxide moieties and 0 or 1 glyceryl moiety, and R₂ and R₃ are each alkyl or monohydroxyalkyl group containing from about 1 to 3 carbon atoms.

(h) Alkyl polysaccharides having a hydrophobic group of 6 to 30, preferably 10, carbon atoms and a polysaccharide group such as glucose, galactose, etc. Suitable alkyl polysaccharides are octyl, nonydecyl, undecyldodecyl, tridecyl, tetradecyl, pentadecyl, hexadecyl, heptadecyl, and octadecyl, di-, tri-, tetra-, penta-, and hexaglucosides, galactosides, lactosides, giucoses, fructosides, fructoses, and so on.

(i) Polyethylene glycol (PEG) glyceryl fatty esters, having the formula
RC(O)OCH₂CH(OH)CH₂(OCH₂CH₂)ₙOH
wherein n is 5-200 and RC(O) is a hydrocarbylcarbonyl group wherein R is preferably an aliphatic radical having 7 to 19 carbon atoms.

(j) Other nonionic surfactants that may be used include C₁₀₋₁₈ alkyl(C₁₋₆)polyhydroxy fatty acid amides such as C₁₂₋₁₈ methylglucamides, N-alkoxy polyhydroxy fatty acid amides, N-propyl through N-hexyl C₁₂₋₁₉ glucamides and so on.

Amphoteric Surfactants

Amphoteric surfactants that can be used in the compositions of the invention are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate.

Suitable amphoteric surfactants may be imidazolinium compounds. Examples of such materials are marketed under the tradename MIRANOL, by Miranol, Inc.

Also suitable amphoteric surfactants are monocarboxylates or dicarboxylates such as cocamphocarboxypropionate, cocoamphocarboxypropionic acid, cocamphocarboxyglycinate, and cocoamphoacetate.

Other types of amphoteric surfactants include aminoalkanoates of the formula
R—NH(CH₂)ₙCOOM
or iminodialkanoates of the formula:
R—N[(CH₂)ₘCOOM]₂
and mixtures thereof; wherein n and m are 1 to 4, R is C₈₋₂₂ alkyl or alkenyl, and M is hydrogen, alkali metal, alkaline earth metal, ammonium or alkanolammonium. Examples of such amphoteric surfactants include n-alkylaminopropionates and n-alkyliminodipropionates, which are sold under the trade name MIRATAINE by Miranol, Inc. or DERIPHAT by Henkel, for example N-lauryl-beta-amino propionic acid, N-lauryl-beta-imino-dipropionic acid, or mixtures thereof.

Zwitterionic surfactants are also suitable for use in the compositions of the invention. Zwitterionics include betaines, for example higher alkyl betaines such as coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxylethyl betaine, and mixtures thereof. Also suitable are sulfo- and amido-betaines such as coco dimethyl sulfopropyl betaine, stearyl dimethyl sulfopropyl betaine, and the like.

Cationic surfactants and/or polymers may be incorporated into the compositions of the invention. If so, 0.01-15%, preferably 0.05-10%, preferably 0.10-8% of a cationic ingredients is suggested. Suitable cationic ingredients include cationic polymers, quaternary ammonium salts, or the salts of fatty amines. Suitable quaternary ammonium compounds may be mono-long chain alkyl, di-long chain alkyl, tri-long chain alkyl, and the like. Examples of such quaternary ammonium salts include behenalkonium chloride, behentrimonium chloride, behentrimonium methosulfate, benzalkonium chloride, benzethonium chloride, benzyl triethyl ammonium chloride, cetalkonium chloride, cetrimonium chloride, cetrimonium bromide, cetrimonium methosulfate, cetrimonium tosylate, cetylpyridinium chloride, dibehenyudiarachidyl dimonium chloride, dibehenyldimonium chloride, dibehenyldimonium methosulfate, dicapryl/dicaprylyl dimonium chloride, dicetyldimonium chloride, and mixtures thereof. Other quaternary ammonium salts useful as the cationic surfactant are salts of fatty primary, secondary, or tertiary amines, wherein the substituted groups have 12 to 22 carbon atoms. Examples of such amines include dimethyl stearamine, dimethyl soyamine, stearylamine, myristylamine, tridecylamine, ethyl stearamine, and so on.

Also suitable as the cationic ingredient are cationic polymers such as:

(a) Quaternary derivatives of cellulose ethers such as polymers sold under the tradename JR-125, JR-400, JR-30M. Preferred is Polyquaternium 10, which is a polymeric quaternary ammonium salt of hydroxyethyl cellulose reacted with a trimethyl ammonium subsituted epoxide.

(b) Copolymers of vinylpyrrolidone.

(c) Homopolymer of dimethyldiallylammonium chloride, or copolymer of dimethyldiallylammonium chloride and acrylamide. Such compounds are sold under the tradename MERQUAT.TM. by Merck and Company.

(d) Homopolymers or copolymers derived from acrylic or methacrylic acid wherein the monomer units are selected from the group consisting of acrylamide, methylacrylamide, diacetone-acrylamide, acrylamide or methacrylamide substituted on the nitrogen by lower alkyl, alkyl esters of acrylic acid and methacrylic acid, vinylpyrrolidone, and vinyl esters.

Examples of cationic polymers that can be used in the compositions of the invention are the cationic polymers disclosed in U.S. Pat. Nos. 5,240,450 and 5,573,709, which are hereby incorporated by reference.

Waxes

The compositions of the invention may contain 0.1-25%, preferably 0.5-20%, more preferably 1-15% by weight of the total composition of wax. Suitable waxes have a melting point of 35 to 120° C., and can be animal waxes, plant waxes, mineral waxes, silicone waxes, synthetic waxes, and petroleum waxes. Examples of waxes in accordance with the invention include bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, PEG-6 beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, ethylene homo- or copolymers, stearoxy dimethicone, dimethicone behenate, stearyl dimethicone, and the like, as well synthetic homo- and copolymer waxes such as PVP/eicosene copolymer, PVP/hexadecene copolymer, and the like.

Branched Chain Silicone Resins

It may be desirable to include one or more branched chain silicone resins in the compositions of the invention. If so, a range of 0.001-20%, preferably 0.01-15%, more preferably 0.1-10%0/by weight of the total composition is suggested. Examples of suitable silicone resins include siloxy silicate polymers having the following general formula:
[(RR′R″)₃SiO_{1/2}]ₓ[SiO₂]_{y}
wherein R, R′ and R″ are each independently a C₁₋₁₀ straight or branched chain alkyl or phenyl, and x and y are such that the ratio of (RR′R″)₃SiO_{1/2} units to SiO₂ units is 0.5 to 1 to 1.5 to 1.

Preferably R, R′ and R″ are a C₁₋₆ alkyl, and more preferably are methyl and x and y are such that the ratio of (CH₃)₃SiO_{1/2} units to SiO₂ units is 0.75 to 1. Most preferred is this trimethylsiloxy silicate containing 2.4 to 2.9 weight percent hydroxyl groups which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxy silicate as described is available from Dow Corning Corporation under the tradename 2-0749 and 2-0747, which is a blend of about 40-60% volatile silicone and 40-60% trimethylsiloxy silicate. Dow Corning 2-0749 in particular, is a fluid containing about 50% trimethylsiloxy silicate and about 50% cyclomethicone. The fluid has a viscosity of 200-700 centipoise at 25° C., a specific gravity of 1.00 to 1.10 at 25° C., and a refractive index of 1.40-1.41.

Other branched chain silicone resins are silicone esters comprising units of the general formula RₐR^{E}_{b}SiO_{[4-(a+b)/2]} or R¹³ₓR^{E}_{y}SiO_{1/2}, wherein R and R¹³ are each independently an organic radical such as alkyl, cycloalkyl, or aryl, or, for example, methyl, ethyl, propyl, hexyl, octyl, decyl, aryl, cyclohexyl, and the like a is an number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred RE radicals are those wherein the ester group is formed of one or more fatty acid moieities (e.g. of about 6, often about 6 to 30 carbon atoms) and one or more aliphatic alcohol moieities (e.g. of about 10 to 30 carbon atoms). Examples of such acid moieities include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxypropane. Preferably the ester subgroup (i.e. the group containing the carboxylic ester) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety. More particularly, the cross-linked silicone ester can be a liquid or solid at room temperature. Preferably it will have a waxy feel and a molecular weight of no more than about 100,000 daltons.

Such silicone resins having the above formula are disclosed in U.S. Pat. No. 4,725,658 and U.S. Pat. No. 5,334,737, which are hereby incorporated by reference. These ingredients are commercially available from General Electric under the tradenames SF 1318 and SF 1312, respectively.

Pigments and Powders

The composition of the invention may contain 0.001-35%, preferably 0.01-20% more preferably 0.1-10%, by weight of the total composition, of dry particulate matter having a particle size of 0.02 to 200, preferably 0.5 to 100, microns. The particulate matter may be colored or non-colored (for example white). Suitable powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone oil or various other agents either alone or in combination, which coat the powder surface and render the particles more lipophilic in nature.

The powder component also may comprise various organic and inorganic pigments. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

The composition may contain a mixture of both pigmented and non-pigmented powders. The percentage of pigments used in the powder component will depend on the type of cosmetic being formulated.

Sunscreens

The compositions of the invention may contain 0.001-20%, preferably 0.01-10%, more preferably 0.05-8% of one or more sunscreens. A sunscreen is defined as an ingredient that absorbs at least 85 percent of the light in the UV range at wavelengths from 290 to 320 nanometers, but transmit UV light at wavelengths longer than 320 nanometers. Sunscreens generally work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorporated by reference. Examples of such sunscreen materials are p-aminobenzoic acid (PABA), cinoxate, diethanolamine p-methoxycinnamate (DEA-methoxycinnamate), Digalloyl trioleate, dioxybenzone (Benzophenone-8), ethyl 4-[bis-(hydroxypropyl)]amnobenzoate(ethyl dihydroxypropyl PABA), 2-ethythexyl-2-cyano-3,3-diphenylacrylate(octocrylene), ethylhexyl p-methoxycinnamate (Octyl methoxycinnamate), 2-ethylhexyl salicylate (Octyl salicylate), glyceryl aminobenzoate (Glyceryl PABA), homosalate, lawsone with dihydroxyacetone, menthyl anthranilate, oxybenzone (Benzophenone-3), Padimate A (Pentyl Dimethyl PABA), Padimate 0, (Octyl Dimethyl PABA), 2-Phenylbenzimidazole-5-sulfonic acid (Phenylbenzimidazole Sulfonic acid), Red Petrolatum, Sulisobenzone (Benzophenone4), triethanolamine salicylate (TEA-Salicylates), and so on.

Preservatives

The composition may contain 0.0001-8%, preferably 0.001-6%, more preferably 0.005-5% by weight of the total composition of preservatives. A variety of preservatives are suitable, including such as benzoic acid, benzyl alcohol, benzylhemiformal, benzylparaben, 5-bromo-5-nitro-1,3-diox-ane, 2-bromo-2-nitropropane-1,3-diol, butyl paraben, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

Alpha or Beta Hydroxy Acids, Alpha Keto Acids

It may be desired to add one or more alpha or beta hydroxy acids or alpha ketoacids to the compositions of the invention. Suggested ranges are 0.01-20%, preferably 0.1-15%, more preferably 0.5-10% by weight of the total composition. Suitable alpha hydroxy acids and alpha ketoacids are disclosed in U.S. Pat. No. 5,091,171, which is hereby incorporated by reference. Such alpha hydroxy acids are as follows:

a) Organic carboxylic acids where one hydroxyl group is attached to the alpha carbon atom of the acid. The general structure of such alpha hydroxy acids may be represented by the following formula:
(Ra)(Rb)C(OH)COOH
wherein Ra and Rb are H, F, Cl, Br, alkyl, aralkyl, or aryl group of saturated, unsaturated, straight or branched chain or cyclic form having 1-10 carbon atoms, and in addition Ra or Rb may carry OH, CHO, COOH and alkoxy groups having 1 to 9 carbon atoms.

The second group of alpha hydroxy acids may be represented by the following formula:
(Ra)COCOO(Rb)
wherein Ra and Rb are H, alkyl, aralkyl, or aryl groups of straight or branched chain saturated or unsaturated alkyl having 1 to 10 carbon atoms, and in addition Ra may carry F, Cl, Br, I, OH, CHO, COOH, and alkoxy groups having 1 to 10 carbon atoms.

The alpha hydroxy acids may exist in the keto acid form, or the ester form. Examples of such alpha hydroxy acids include glycolic acid, malic acid, pyruvic acid, mandelic acid, lactic acid, methyllactic acid, and so on.

Also beta hydroxy acids such as salicylic acid, and derivatives thereof may be included in the compositions of the invention.

The anhydrous emulsions of the invention are made using conventional techniques known by those skilled in the art of cosmetic formulation.

The composition containing the ascorbic acid, nonaqueous polar organic solvent and nonaqueous nonpolar organic solvent will typically be applied onto marionette lines for a period of time sufficient to effectuate a statistically significant improvement in their appearance.

The present invention will be better understood from the examples which follow, all of which are intended for illustrative purposes only and are not meant to unduly limit the scope of the invention in any way.

### EXAMPLE 1

A formulation for treating marionette lines in accordance with the present invention is listed in Table 1, below.

TABLE 1

| Ingredient | % by weight |
|---|---|
| Propylene glycol | 45.00 |
| Ascorbic acid | 10.50 |
| glycerin | 7.00 |
| adenosine | 0.04 |
| Cetyl PEG/PPG - 10/1 | 3.00 |
| dimethicone | |
| Cyclopentasiloxane and | 18.00 |
| dimethicone crosspolymer | |
| cyclopentasiloxane | 15.50 |
| Acrylates copolymer | 0.46 |
| Lauroyl lysine | 0.50 |
| | 100.00 |

The above-identified composition was then evaluated in order to determine its effect on treating marionette lines. The results are found in Table 2, below.

TABLE 2

| | SAMPLE | | FOUR (4) | EIGHT (8) | TWELVE (12) | P- |
|---|---|---|---|---|---|---|
| ATTRIBUTE | SIZE | BASELINE | WEEKS | WEEKS | WEEKS | VALUE |
| Marionette | 54 | 3.50 | 3.00 | 3.00 | 3.00 | <0.001 |
| Lines | | (2.00-4.00) | (2.00-4.00) | (2.00-4.00) | (2.00-4.00) | |

As can be seen from the results, treatment of marionette lines with the composition in Table 1 yielded a statistically significant frequency of improvement in reducing marionette line appearance.

These results were compared to treatment of marionette lines with a conventional retinol-based cream (0.075% retinol), the results of which are found in Table 3, below.

TABLE 3

| | SAMPLE | | FOUR (4) | EIGHT (8) | TWELVE (12) | P- |
|---|---|---|---|---|---|---|
| ATTRIBUTE | SIZE | BASELINE | WEEKS | WEEKS | WEEKS | VALUE |
| Marionette | 54 | 3.50 | 3.50 | 3.50 | 3.00 | 0.135 |
| Lines | | (2.00-4.00) | (2.00-4.00) | (2.00-4.00) | (2.00-4.00) | |

As can be seen from the results in Table 3., treatment of marionette lines with a composition containing 0.075% conc. of retinol failed to yield a statistically significant frequency of improvement in reducing marionette line appearance.

What is claimed is:

1. A process for treating marionette lines damaged by age, sun exposure and pollution comprising contacting the marionette lines with a composition containing:
(a) from about 1 to about 20% by weight of ascorbic acid;
(b) from about 30 to about 80% by weight of a nonaqueous polar organic solvent; and
(c) from about 20 to about 60% by weight of a nonaqueous nonpolar organic solvent, all weights being based on the weight of the composition.

2. The process of claim 1 wherein the composition is anhydrous.

3. The process of claim 1 wherein the ascorbic acid is present in the composition in an amount of from about 5 to about 15% by weight, based on the weight of the composition.

4. The process of claim 1 wherein the nonaqueous polar organic solvent is present in the composition in an amount of from about 40 to about 60% by weight, based on the weight of the composition.

5. The process of claim 1 wherein the nonaqueous polar organic solvent is chosen from monohydric alcohols and dihydric alcohols.

6. The process of claim 1 wherein the nonaqueous polar organic solvent is propylene glycol.

7. The process of claim 1 wherein the nonaqueous nonpolar organic solvent is present in the composition in an amount of from about 20 to about 50% by weight, based on the weight of the composition.

8. The process of claim 1 wherein the nonaqueous nonpolar organic solvent is chosen from at least one silicone.

9. A process for imparting a more youthful appearance onto a human face comprising contacting marionette lines present on the human face with a composition containing:
(a) from about 1 to about 20% by weight of ascorbic acid;
(b) from about 30 to about 80% by weight of a nonaqueous polar organic solvent; and
(c) from about 20 to about 60% by weight of a nonaqueous nonpolar organic solvent, all weights being based on the weight of the composition.
